(19) United States
(12) Patent Application Publication
(10) Pub. No.: US 2023/0169655 A1
(43) Pub. Date: Jun. 1, 2023

(54) Orthopedic Surgical Planning System with Automated Bone Density Measurement

(71) Applicants: OsteoApp.ai, Inc., Rochester, MN (US); Mayo Foundation for Medical Education and Research, Rochester, MN (US)

(72) Inventors: Peter T. Bianco, Rochester, NY (US); Joshua Jacobs, Rochester, MN (US); Jo De Schepper, Sinaai (BE); Sanjeev Kakar, Rochester, MN (US)

(21) Appl. No.: 18/059,796

(22) Filed: Nov. 29, 2022

Related U.S. Application Data
(60) Provisional application No. 63/283,765, filed on Nov. 29, 2021.

(51) Int. Cl. G06T 7/00; G16H 50/30; A61B 5/00; A61B 6/00

(52) U.S. Cl. CPC G06T 7/0012; G16H 50/30; A61B 5/4509; A61B 6/505; G06T 2207/30008; A61B 2034/102

(57) ABSTRACT

Techniques are described herein for automated bone mineral density and fracture risk assessment from digital X-rays. A method includes: obtaining image data including information relating to cortical bone tissue of at least a part of a first bone; preprocessing the image data, by isolating a region of interest in the image data, to generate preprocessed image data; applying the preprocessed image data as inputs across a trained machine learning model to generate output indicative of bone mineral density of the first bone; determining a T-score value based on the bone mineral density of the first bone; and providing, on a user interface, an output based on the T-score value.

### BACKGROUND

A total hip arthroplasty procedure is a surgical procedure in which the hip joint is replaced by a prosthetic implant. Total hip arthroplasty may be performed in cases where a patient has severe degenerative arthritis, osteonecrosis, subcapital femoral neck fractures, or severe hip pain caused by other conditions of the hip.

Prior to replacing a patient's hip joint with a prosthetic implant, a surgeon may select a particular size and/or type of prosthetic implant to use in a total hip arthroplasty procedure based on the surgeon's evaluation of the patient's femur, including an identified level of osteoporosis (if any). For example, a surgeon may evaluate the patient's proximal femur using the Dorr classification, and the surgeon may select the type of prosthetic implant and implant fixation type based on the surgeon's determination of the Dorr type of the femur.

Using the Dorr classification, a surgeon may qualitatively classify a “champagne flute” shaped femur having a narrow canal with thick cortical walls as a Dorr Type A femur. A surgeon may classify a femur having moderate cortical walls as a Dorr Type B femur. A surgeon may classify a “stovepipe” shaped femur having thin cortical walls as a Dorr Type C femur. Quantitative assessment techniques may also be used by digital measurement of various anatomic landmarks and bony structures of the proximal femoral geometries.

Different surgeons may classify a particular femur into different Dorr types based upon differing interpretations of qualitative factors. Accordingly, the reproducibility of the Dorr classification may be reduced as a result of differing interpretations of qualitative factors by different surgeons, and some potentially osteoporotic femurs may not be identified prior to a total hip arthroplasty procedure. Quantitative measurements of the Dorr index are often difficult due to varying magnifications of the X-ray and human factors.

In cases where a patient's femur is misclassified (e.g., a patient has an osteoporotic femur that is not identified) prior to a total hip arthroplasty procedure, a surgeon may not select the type of prosthetic implant and/or use other techniques that minimize the risk of complications such as periprosthetic fractures. A patient may therefore be more susceptible to periprosthetic fractures and other complications than would be the case had a different size and/or type of prosthetic implant been selected and/or had other techniques been used.

Additionally, patients undergoing total hip arthroplasty and/or other orthopedic procedures around the axial and appendicular skeleton that include implant insertion, fracture fixation, joint arthroplasty (including but not exhaustive of spinal, shoulder, elbow, wrist, hand, fingers, knee, ankle, foot, and toes), and/or bony fusion may be undiagnosed with osteoporosis, placing them at risk for fracture complications, hardware failure, malunions, non-unions, delayed unions, functional sequelae, and hospital readmissions. While a doctor may infer subjective bone strength prior to performing a procedure, a doctor may be unable to quantitatively evaluate bone mineral density (BMD) during preoperative planning.

### SUMMARY

Implementations described herein relate to various systems, devices, and techniques for providing preoperative planning for orthopedic procedures that include implant insertion, joint arthroplasty (including but not exhaustive of spinal, shoulder, elbow, wrist, hand, fingers, knee, ankle, foot, and toes) fracture fixation, and/or bony fusion (e.g., total hip arthroplasty, joint arthroplasty, and fracture fixation including spine, hip, and wrist fractures and joint fusions, etc.), including providing adjunct automated bone mineral density and fracture risk assessment from digital X-rays.

Pre-operative planning for orthopedic procedures that include implant insertion, fracture fixation, and/or bony fusion may include computer-assisted and/or manual techniques for determining implant sizing and position, restoration of normal mechanical function, resulting in pain relief and/or implant longevity. The computer-assisted and/or manual pre-operative planning techniques provided according to various implementations may be used in joint arthroplasty, fracture fixation (e.g. spine, hip, and wrist fractures, etc.), and joint fusions, among others.

As an example, osteoporosis status of a patient undergoing total hip replacement may be a significant factor in the long term success of a total hip arthroplasty procedure. With conventional pre-operative planning techniques, nearly 25% of patients undergoing Total Joint Replacement surgery may be undiagnosed with osteoporosis, placing them at risk for fracture complications and hospital readmissions. Bone Mineral Density (BMD) is a measurement that may provide useful information to orthopedic surgeons performing hip replacement surgery or any other orthopedic procedures as noted above. In particular, pre-operative identification of BMD may prevent or reduce complications (e.g., periprosthetic fractures). However, conventional pre-operative planning techniques typically do not provide a quantitative BMD assessment.

Some of these problems with conventional pre-operative planning techniques may be addressed by implementations, which may provide an automated method for conveniently obtaining an accurate BMD score and subsequent fracture risk assessment from standard X-rays during the total hip replacement pre-operative planning process. By obtaining patient-specific osteoporosis related fracture risk assessment prior to the actual surgery, the surgeon can assess the need to alter surgical technique, implant selection, and post-operative patient management and rehabilitation based on the severity of the patient's BMD status.

In some implementations, a computer-based system and method may identify quantitative indices of geometric landmarks in a human femur and algorithmically sort these femurs into classified geometries according to the Dorr Classification system, for example, for femoral geometry. The Dorr classification system is a system for inferring subjective bone strength from femoral geometry. Further, auto segmentation of various femoral geometries may be applied to the patient-specific X-ray. These segmentations may be used to isolate areas of interest in the patient femur such that a trained convolutional neural network (CNN) algorithm or vision transformers (ViT) can be used to measure and assess fracture risk assessment and return a T-Score and Z-score report. In some implementations, such a report may be returned within seconds. The CNN may be trained using retrospective geometrical indices measurement of preoperative hip arthroplasty or other hip X-rays of patients who also had a concomitant dual-energy X-ray absorptiometry (DXA) osteoporosis examination within, e.g., one year of the original hip X-ray. Patient-specific DXA results paired with the patient-specific hip X-ray may be used as the ground truth for the CNN algorithm.

In some implementations, a use case may include a patient receiving a series of digital X-rays (A-P and Lateral views) in the physician clinic or another facility leading to a diagnosis of osteoarthritis and indicated for total hip replacement. Once the images are obtained, they may be electronically transferred from the facility picture archiving and communication system (PACS) system to cloud-based servers where the algorithmic assessment is completed, and results may be sent back to the physician clinic or another facility (e.g., within seconds). The results may be displayed with the original X-ray image on the clinic workstation, provider smartphone, tablet, etc. Sending the image for analysis can take place at the time of initial exam and diagnosis, or prior to surgery during the preoperative planning process where implant selection, implant sizing and instrumentation requirements are determined for carrying out the surgery.

In some implementations, a computer-based system and method provides implant choice recommendation(s) and/or and treatment recommendation(s), during preoperative planning, to reduce a patient's risk of complications including periprosthetic fractures or implant loosening. In some implementations, the computer-based system and method may make these implant and/or treatment recommendations based on standard full, bi-lateral anterior-posterior (AP) pelvis X-ray views (e.g., of the unilateral affected hip). The affected hip may be osteopenic or osteoporotic (e.g., due to disuse because of osteoarthritic pain). In some implementations, a localized view of bone quality is determined, from which the implant recommendation may then be made. In some implementations, a T-score of the contralateral (non-affected) hip may also be determined, to provide a broader view of bone quality for a patient. In some implementations, based on this broader view of bone quality, the system may also provide treatment recommendation(s), potentially leading to a broader osteoporotic care pathway for the patient following the orthopedic implant procedure.

In some implementations, the system may be used in the treatment of orthopedic trauma patients, especially those presenting with hip fractures, pubic rami fractures, and periprosthetic hip fractures. A pelvic X-ray may be taken in these cases and used by the system to algorithmically assess the non-affected hip. In some implementations, the system may identify those at high risk for subsequent secondary hip fracture, based on the pelvic X-ray and provide treatment recommendations for potential intervention (e.g., prophylactic treatment of the non-affected hip during fracture repair surgery). In addition, the system may provide a baseline BMD measurement that may be used for further decision making in osteoporotic care (e.g., to guide future clinical decisions in preventing secondary fractures).

In some implementations, the system may be used to determine BMD in, e.g., postmenopausal women with a history of hip pain and other patients who come through clinics (e.g., orthopedic surgeon, rehab doctor, rheumatologist, endocrinologist, etc.). Initial investigation for these complaints may include history taking, a clinical exam, and a baseline X-ray of pelvis and painful hip, and potentially an ultrasound. In some implementations, the system may utilize the X-rays taken during the initial investigation and process them using artificial intelligence techniques to determine BMD and identify osteoporosis in previously undiagnosed patients. Accordingly, in some implementations, the system may be used as a diagnostic and treatment tool to identify osteoporosis and provide a treatment plan to strengthen bone and/or prevent secondary fractures during or after surgery. In some implementations, the treatment plan may include pre-surgical treatment (e.g., injections to strengthen bone prior to surgery) and post-surgical treatment, as well as a determination of implant type and/or size and/or other treatments or techniques to be used during surgery.

The above description is provided as an overview of some implementations of the present disclosure. Further description of those implementations, and other implementations, are described in more detail below.

Various implementations can include a non-transitory computer readable storage medium storing instructions executable by one or more processors (e.g., central processing unit(s) (CPU(s)), graphics processing unit(s) (GPU(s)), digital signal processor(s) (DSP(s)), and/or tensor processing unit(s) (TPU(s)) to perform a method such as one or more of the methods described herein. Other implementations can include a client device (e.g., a client device including at least an interface for interfacing with cloud-based component(s)) that includes processor(s) operable to execute stored instructions to perform a method, such as one or more of the methods described herein. Yet other implementations can include a system of one or more servers that include one or more processors operable to execute stored instructions to perform a method such as one or more of the methods described herein.

### BRIEF DESCRIPTION OF THE DRAWINGS

FIG. 1 depicts an example environment in which selected aspects of the present disclosure may be implemented, in accordance with various implementations.

FIG. 2 depicts a flowchart illustrating an example method for practicing selected aspects of the present disclosure.

FIG. 3 depicts an example architecture of a computing device.

FIG. 4 depicts an example of training a convolutional neural network algorithm.

FIG. 5 depicts an example of bone mineral density determination and treatment, in accordance with various implementations.

FIG. 6 depicts an example of using a T-score to select an implant and treatment, in accordance with various implementations.

### DETAILED DESCRIPTION

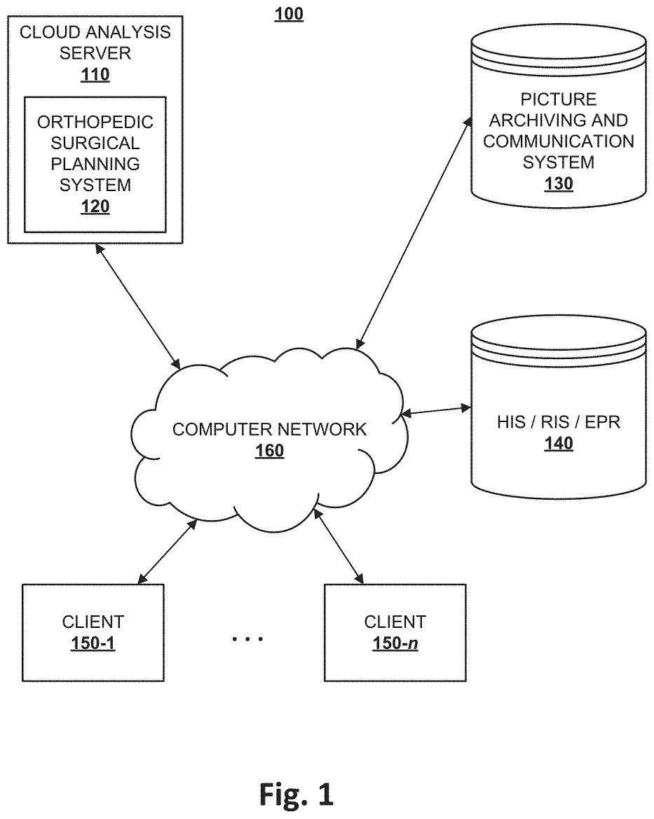

FIG. 1 schematically depicts an example environment 100 in which selected aspects of the present disclosure may be implemented, in accordance with various implementations. Any computing devices depicted in FIG. 1 or elsewhere in the figures may include logic such as one or more microprocessors (e.g., central processing units or “CPUs”, graphical processing units or “GPUs”) that execute computer-readable instructions stored in memory, or other types of logic such as application-specific integrated circuits (“ASIC”), field-programmable gate arrays (“FPGA”), and so forth. Some of the systems depicted in FIG. 1, such as a cloud analysis server 110, may be implemented using one or more server computing devices that form what is sometimes referred to as a “cloud infrastructure,” although this is not required.

In implementations, the environment 100 may include a cloud analysis server 110 that includes an orthopedic surgical planning system 120 that implements an orthopedic surgical planning application that is accessible from various clients, including clients 150-1, . . . , 150-n that may be included in the environment 100, through either a thin client interface, such as a web browser (e.g., a web-based orthopedic surgical planning application), or a program interface. In implementations, the orthopedic surgical planning application that is implemented by the orthopedic surgical planning system 120 may be a software as a service (SaaS) orthopedic surgical planning application. The orthopedic surgical planning system 120 and the clients 150-1, . . . , 150-n may be in communication via a computer network 160, which may be any suitable network including any combination of a local area network (LAN), wide area network (WAN), or the Internet. The orthopedic surgical planning system 120 may be configured to perform selected aspects of the present disclosure in order to determine an accurate BMD score and subsequent fracture risk assessment from X-rays and/or to determine treatments to minimize fracture risk.

Each of the clients 150-1, . . . , 150-n may be, for example, a user computing device that is used by a user to access an orthopedic surgical planning application via an orthopedic surgical planning application user interface, such as a SaaS orthopedic surgical planning application, that is provided by the orthopedic surgical planning system 120, e.g., through a web browser. In an example, the clients 150-1, . . . , 150-n may be user computing devices associated with an individual or an entity or organization such as a hospital, doctor's office, clinic, etc. or any other organization that uses an orthopedic surgical planning application. For example, a surgeon may operate an orthopedic surgical planning application to determine a patient's BMD score, to perform a fracture risk assessment, and/or to determine an appropriate treatment and treat a patient.

In various implementations, the environment 100 may include picture archiving and communication system 130, which may store patient X-ray images that may be obtained and analyzed by the orthopedic surgical planning system 120 to determine a patient's BMD score. The picture archiving and communication system 130 may be implemented using one or more server computing devices that form what is sometimes referred to as a “cloud infrastructure,” although this is not required. The orthopedic surgical planning system 120 and the picture archiving and communication system 130 may be in communication via the computer network 160.

In various implementations, the environment 100 may include hospital information system (HIS)/radiology information system (RIS)/electronic patient records (EPR) 140, which may store patient records that may be obtained, utilized, and updated by the orthopedic surgical planning system 120. The HIS/RIS/EPR 140 may be implemented using one or more server computing devices that form what is sometimes referred to as a “cloud infrastructure,” although this is not required. The orthopedic surgical planning system 120 and the HIS/RIS/EPR 140 may be in communication via the computer network 160.

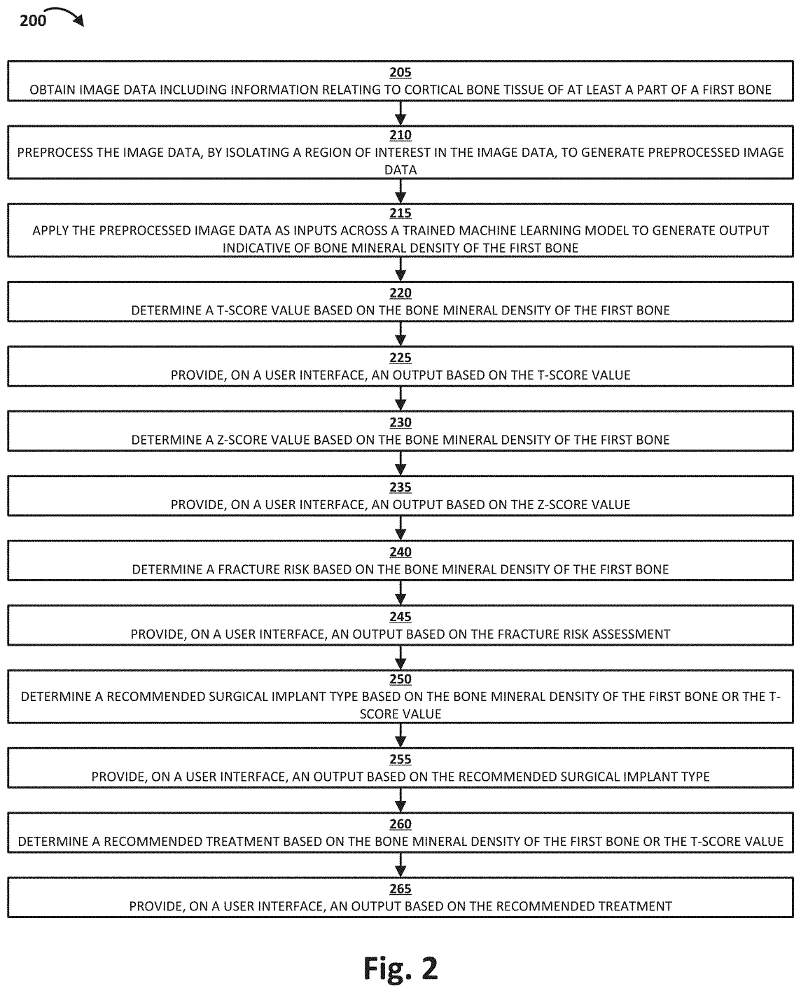

FIG. 2 is a flowchart illustrating an example method 200 of automated bone mineral density and fracture risk assessment, in accordance with implementations disclosed herein. For convenience, the operations of the flowchart are described with reference to a system that performs the operations. This system may include various components of various computer systems, such as one or more components of the cloud analysis server 110. Moreover, while operations of method 200 are shown in a particular order, this is not meant to be limiting. One or more operations may be reordered, omitted, or added.

At block 205, the system may obtain image data including information relating to cortical bone tissue of at least a part of a first bone. In implementations, at block 205, the orthopedic surgical planning system 120 of the cloud analysis server 110 may obtain image data including information relating to cortical bone tissue of at least a part of a first bone, e.g., from the picture archiving an communication system 130. In an example, responsive to a request from one of the clients 150-1, . . . , 150-n to perform a BMD analysis and/or fracture risk assessment for a particular patient having an electronic patient record in the HIS/RIS/EPR 140, the orthopedic surgical planning system 120 may obtain portions of the electronic patient record for the patient from the HIS/RIS/EPR 140 and may obtain X-ray image data for the patient from the picture archiving and communication system 130.

Still referring to FIG. 2, at block 210, the system may preprocess the image data, by isolating a region of interest in the image data, to generate preprocessed image data. In implementations, at block 210, the orthopedic surgical planning system 120 of the cloud analysis server 110 may preprocess the image data obtained at block 205, to generate preprocessed image data. In some implementations, the orthopedic surgical planning system 120 may pre-process the image data by co-registering it to a common space, isolating regions of interest (e.g., a single femur), and optionally performing image segmentation.

Still referring to FIG. 2, at block 215, the system may apply the preprocessed image data as inputs across a trained machine learning model to generate output indicative of bone mineral density of the first bone. In implementations, at block 215, the orthopedic surgical planning system 120 of the cloud analysis server 110 may apply the preprocessed image data generated at block 210 as inputs across a trained machine learning model to generate output indicative of bone mineral density of the first bone. The trained machine learning model may be a convolutional neural network. In other implementations, at block 215, the orthopedic surgical planning system 120 of the cloud analysis server 110 may use vision transformers to generate output indicative of bone mineral density of the first bone.

Still referring to FIG. 2, at block 220, the system may determine a T-score value based on the bone mineral density of the first bone. In implementations, at block 220, the orthopedic surgical planning system 120 of the cloud analysis server 110 may determine a T-score value based on the bone mineral density of the first bone, determined based on the output of the trained machine learning model at block 215. In addition to using the bone mineral density of the first bone, the orthopedic surgical planning system 120 of the cloud analysis server 110 may use demographic information (e.g., ethnicity, race, etc.) to determine the T-score value.

Still referring to FIG. 2, at block 225, the system may provide, on a user interface, an output based on the T-score value. In implementations, at block 225, the orthopedic surgical planning system 120 of the cloud analysis server 110 may provide, on a user interface of the client 150-1, . . . , 150-n that requested the BMD analysis and/or fracture risk assessment for the particular patient, an output based on the T-score value determined at block 220.

Still referring to FIG. 2, at block 230, the system may determine a Z-score value based on the bone mineral density of the first bone. In implementations, at block 230, the orthopedic surgical planning system 120 of the cloud analysis server 110 may determine a Z-score value based on the bone mineral density of the first bone determined based on the output of the trained machine learning model at block 215.

Still referring to FIG. 2, at block 235, the system may provide, on a user interface, an output based on the Z-score value. In implementations, at block 235, the orthopedic surgical planning system 120 of the cloud analysis server 110 may provide, on a user interface of the client 150-1, . . . , 150-n that requested the BMD analysis and/or fracture risk assessment for the particular patient, an output based on the Z-score value determined at block 230.

Still referring to FIG. 2, at block 240, the system may determine a fracture risk based on the bone mineral density of the first bone. In implementations, at block 240, the orthopedic surgical planning system 120 of the cloud analysis server 110 may determine a fracture risk based on the bone mineral density of the first bone determined based on the output of the trained machine learning model at block 215.

Still referring to FIG. 2, at block 245, the system may provide, on a user interface, an output based on the fracture risk assessment. In implementations, at block 245, the orthopedic surgical planning system 120 of the cloud analysis server 110 may provide, on a user interface of the client 150-1, . . . , 150-n that requested the BMD analysis and/or fracture risk assessment for the particular patient, an output based on the fracture risk assessment determined at block 240.

Still referring to FIG. 2, at block 250, the system may determine a recommended surgical implant type based on the bone mineral density of the first bone or the T-score value. In implementations, at block 250, the orthopedic surgical planning system 120 of the cloud analysis server 110 may determine a recommended surgical implant type based on the bone mineral density of the first bone determined based on the output of the trained machine learning model at block 215 or the T-score value determined at block 220.

Still referring to FIG. 2, at block 255, the system may provide, on a user interface, an output based on the recommended surgical implant type. In implementations, at block 255, the orthopedic surgical planning system 120 of the cloud analysis server 110 may provide, on a user interface of the client 150-1, . . . , 150-n that requested the BMD analysis and/or fracture risk assessment for the particular patient, an output based on the recommended surgical implant type determined at block 250.

Still referring to FIG. 2, at block 260, the system may determine a recommended treatment based on the bone mineral density of the first bone or the T-score value. In implementations, at block 260, the orthopedic surgical planning system 120 of the cloud analysis server 110 may determine a recommended treatment based on the bone mineral density of the first bone determined based on the output of the trained machine learning model at block 215 or the T-score value determined at block 220.

Still referring to block 260, in an example, a first threshold may be −3, and a second threshold may be −1. In response to determining that a femur or other bone has a T-score that satisfies (e.g., is greater than) both the first threshold and the second threshold (e.g., has a T-score that is greater than −1), the orthopedic surgical planning system 120 may recommend no change to surgical protocol, and standard implant selection. In response to determining that a femur or other bone has a T-score that satisfies the first threshold but does not satisfy the second threshold (e.g., has a T-score that is in the range of −1 to −3), the orthopedic surgical planning system 120 may recommend considering implant selection and prophylactic treatment for osteopenia. In response to determining that a femur or other bone has a T-score that does not satisfy the first threshold and does not satisfy the second threshold (e.g., has a T-score that is less than −3), the orthopedic surgical planning system 120 may recommend initiating pharmacologic/orthobiologic intervention and cemented stem and prophylactic intraoperative cerclage wiring. In other implementations, a different number of thresholds may be used (e.g., one threshold, three thresholds, etc.). In other implementations, bone mineral density, Z-score, etc. may be used instead of or in addition to a T-score.

Still referring to FIG. 2, at block 265, the system may provide, on a user interface, an output based on the recommended treatment. In implementations, at block 265, the orthopedic surgical planning system 120 of the cloud analysis server 110 may provide, on a user interface of the client 150-1, . . . , 150-n that requested the BMD analysis and/or fracture risk assessment for the particular patient, an output based on the recommended treatment determined at block 260.

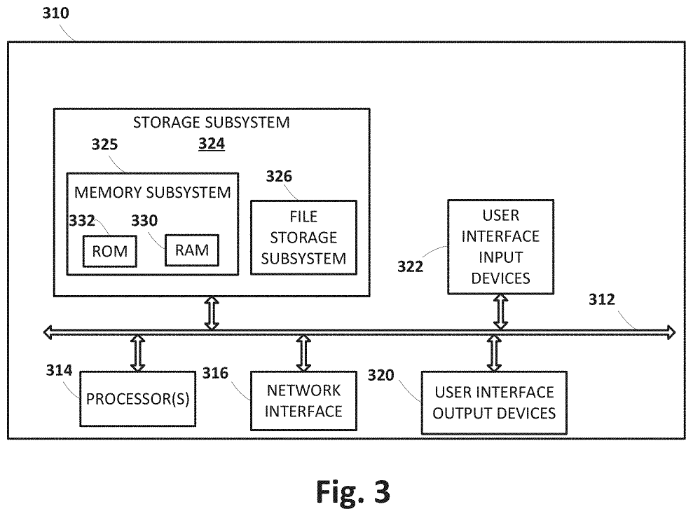

FIG. 3 is a block diagram of an example computing device 310 that may optionally be utilized to perform one or more aspects of techniques described herein. Computing device 310 typically includes at least one processor 314 that communicates with a number of peripheral devices via bus subsystem 312. These peripheral devices may include a storage subsystem 324, including, for example, a memory subsystem 325 and a file storage subsystem 326, user interface output devices 320, user interface input devices 322, and a network interface subsystem 316. The input and output devices allow user interaction with computing device 310. Network interface subsystem 316 provides an interface to outside networks and is coupled to corresponding interface devices in other computing devices.

User interface input devices 322 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computing device 310 or onto a communication network.

User interface output devices 320 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computing device 310 to the user or to another machine or computing device.

Storage subsystem 324 stores programming and data constructs that provide the functionality of some or all of the modules described herein. For example, the storage subsystem 324 may include the logic to perform selected aspects of the methods of FIG. 2, as well as to implement various components depicted in FIG. 1.

These software modules are generally executed by processor 314 alone or in combination with other processors. The memory subsystem 325 included in the storage subsystem 324 can include a number of memories including a main random access memory (RAM) 330 for storage of instructions and data during program execution and a read only memory (ROM) 332 in which fixed instructions are stored. A file storage subsystem 326 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 326 in the storage subsystem 324, or in other machines accessible by the processor(s) 314.

Bus subsystem 312 provides a mechanism for letting the various components and subsystems of computing device 310 communicate with each other as intended. Although bus subsystem 312 is shown schematically as a single bus, alternative implementations of the bus subsystem may use multiple busses.

Computing device 310 can be of varying types including a workstation, server, computing cluster, blade server, server farm, or any other data processing system or computing device. Due to the ever-changing nature of computers and networks, the description of computing device 310 depicted in FIG. 3 is intended only as a specific example for purposes of illustrating some implementations. Many other configurations of computing device 310 are possible having more or fewer components than the computing device depicted in FIG. 3.

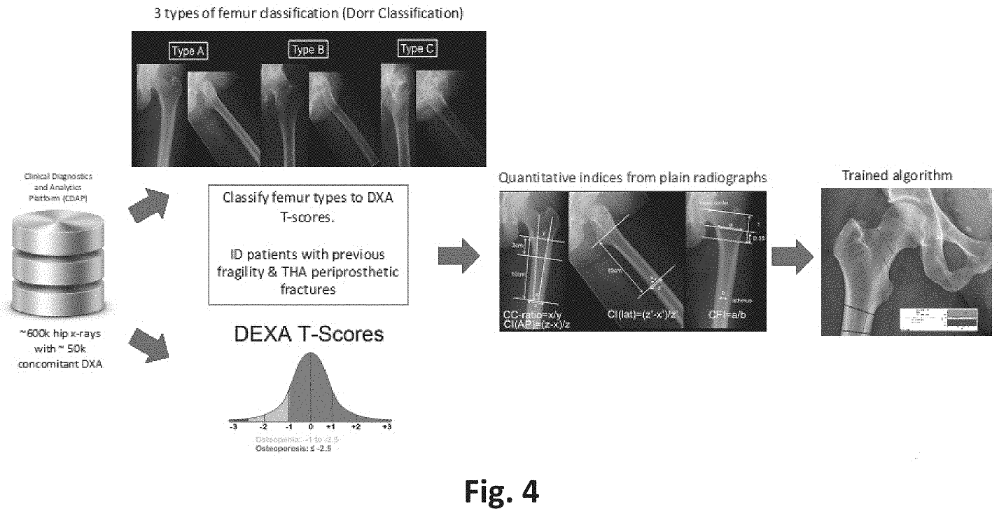

FIG. 4 depicts an example of training a CNN. In some implementations, as illustrated in FIG. 4, the CNN may be trained using retrospective geometrical indices measurement of preoperative hip arthroplasty or other hip x-rays of patients who also had a concomitant dual-energy x-ray absorptiometry (DXA) osteoporosis examination within, e.g., one year of the original hip x-ray. Patient-specific DXA results paired with the patient-specific hip x-ray may be used as the ground truth for the CNN algorithm. In an example, approximately 600,000 hip x-rays may be used to train the CNN, with femur types classified according to DXA T-scores. Patients with previous fragility and THA periprosthetic fractures may be identified. In an example, femurs having T-scores in the range of −1 to −2.5 may be labeled as having osteopenia, and femurs having T-scores less than or equal to −2.5 may be labeled as having osteoporosis.

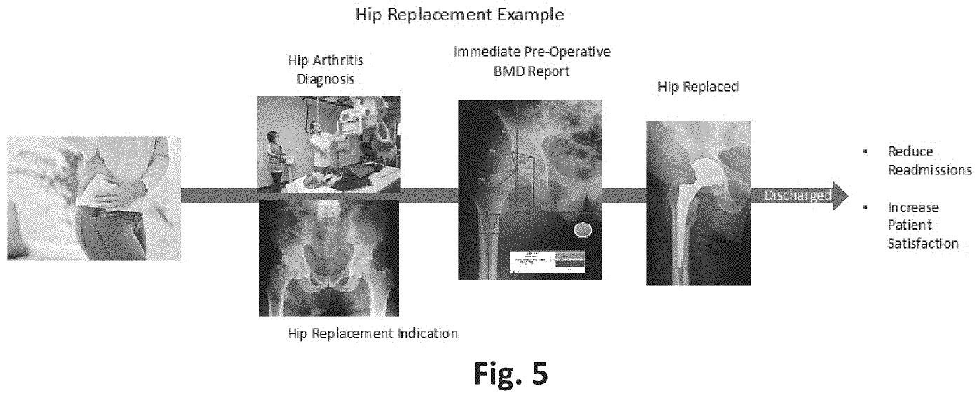

FIG. 5 depicts an example of bone mineral density determination and treatment, in accordance with various implementations. Hip arthritis may be diagnosed, with hip replacement indicated. The orthopedic surgical planning system 120 of FIG. 1 may provide an immediate pre-operative BMD report, and hip replacement may be performed using an implant type, size, and/or position recommended by the orthopedic surgical planning system 120. Patient readmissions may be reduced, and patient satisfaction may be increased.

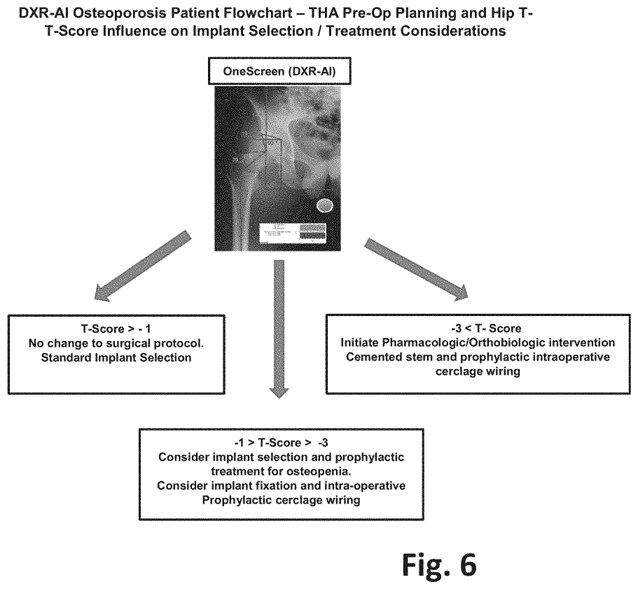

FIG. 6 depicts an example of using a T-score to select an implant and treatment, in accordance with various implementations. In an example, in response to determining that a femur or other bone has a T-score greater than −1, the orthopedic surgical planning system 120 may recommend no change to surgical protocol, and standard implant selection. In response to determining that a femur or other bone has a T-score in the range of −1 to −3, the orthopedic surgical planning system 120 may recommend considering implant selection and prophylactic treatment for osteopenia. In response to determining that a femur or other bone has a T-score less than −3, the orthopedic surgical planning system 120 may recommend initiating pharmacologic/orthobiologic intervention and cemented stem and prophylactic intraoperative cerclage wiring.

While several implementations have been described and illustrated herein, a variety of other means and/or structures for performing the function and/or obtaining the results and/or one or more of the advantages described herein may be utilized, and each of such variations and/or modifications is deemed to be within the scope of the implementations described herein. More generally, all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific implementations described herein. It is, therefore, to be understood that the foregoing implementations are presented by way of example only and that, within the scope of the appended claims and equivalents thereto, implementations may be practiced otherwise than as specifically described and claimed. Implementations of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

What is claimed is:

1. A method implemented by one or more processors, the method comprising:
obtaining image data comprising information relating to cortical bone tissue of at least a part of a first bone;
preprocessing the image data, by isolating a region of interest in the image data, to generate preprocessed image data;
applying the preprocessed image data as inputs across a trained machine learning model to generate output indicative of bone mineral density of the first bone;
determining a T-score value based on the bone mineral density of the first bone; and
providing, on a user interface, an output based on the T-score value.

2. The method according to claim 1, further comprising:
determining a Z-score value based on the bone mineral density of the first bone; and
providing, on the user interface, an output based on the Z-score value.

3. The method according to claim 1, further comprising:
determining a fracture risk based on the bone mineral density of the first bone; and
providing, on the user interface, an output based on the fracture risk assessment.

4. The method according to claim 1, further comprising:
determining a recommended surgical implant type based on the bone mineral density of the first bone or the T-score value; and
providing, on the user interface, an output based on the recommended surgical implant type.

5. The method according to claim 4, wherein determining the recommended surgical implant type based on the bone mineral density of the first bone or the T-score value comprises:
in response to the bone mineral density or the T-score satisfying a first threshold and satisfying a second threshold, determining that the recommended surgical implant type is a first surgical implant type;
in response to the bone mineral density or the T-score satisfying the first threshold but not satisfying the second threshold, determining that the recommended surgical implant type is a second surgical implant type; and
in response to the bone mineral density or the T-score not satisfying the first threshold and not satisfying the second threshold, determining that the recommended surgical implant type is a third surgical implant type.

6. The method according to claim 1, further comprising:
determining a recommended treatment based on the bone mineral density of the first bone or the T-score value; and
providing, on the user interface, an output based on the recommended treatment.

7. The method according to claim 6, wherein determining the recommended treatment based on the bone mineral density of the first bone or the T-score value comprises:
in response to the bone mineral density or the T-score satisfying a first threshold and satisfying a second threshold, determining that the recommended treatment is a first treatment;
in response to the bone mineral density or the T-score satisfying the first threshold but not satisfying the second threshold, determining that the recommended treatment is a second treatment; and
in response to the bone mineral density or the T-score not satisfying the first threshold and not satisfying the second threshold, determining that the recommended treatment is a third treatment.

8. The method according to claim 1, wherein the trained machine learning model is a convolutional neural network or vision transformer.

9. The method according to claim 1, wherein:
the first bone is a femur; and
isolating the region of interest in the image data comprises applying auto-segmentation of various femoral geometries to the image data.

10. The method according to claim 9, wherein applying the preprocessed image data as the inputs across the trained machine learning model further generates output indicative of quantitative indices of geometric landmarks in the femur.

11. The method according to claim 10, further comprising:
determining a Dorr type of the femur based on the quantitative indices of geometric landmarks in the femur; and
providing, on the user interface, an output based on the Dorr type of the femur.

12. The method according to claim 1, wherein:
the trained machine learning model is trained using a set of training images, each training image in the set of training images comprising information relating to cortical bone tissue of at least a part of a bone in the training image; and
each training image in the set of training images is labeled with bone mineral density of the bone in the training image.

13. A computer program product comprising one or more computer-readable storage media having program instructions collectively stored on the one or more computer-readable storage media, the program instructions executable to:
obtain image data comprising information relating to cortical bone tissue of at least a part of a first bone;
preprocess the image data, by isolating a region of interest in the image data, to generate preprocessed image data;
apply the preprocessed image data as inputs across a trained machine learning model to generate output indicative of bone mineral density of the first bone;
determine a T-score value based on the bone mineral density of the first bone; and
provide, on a user interface, an output based on the T-score value.

14. The computer program product according to claim 13, the program instructions further being executable to:
determine a Z-score value based on the bone mineral density of the first bone; and
provide, on the user interface, an output based on the Z-score value.

15. The computer program product according to claim 13, the program instructions further being executable to:
determine a fracture risk based on the bone mineral density of the first bone; and
provide, on the user interface, an output based on the fracture risk assessment.

16. The computer program product according to claim 13, the program instructions further being executable to:
determine a recommended surgical implant type based on the bone mineral density of the first bone or the T-score value; and
provide, on the user interface, an output based on the recommended surgical implant type.

17. A system comprising:
a processor, a computer-readable memory, one or more computer-readable storage media, and program instructions collectively stored on the one or more computer-readable storage media, the program instructions executable to:
obtain image data comprising information relating to cortical bone tissue of at least a part of a first bone;
preprocess the image data, by isolating a region of interest in the image data, to generate preprocessed image data;
apply the preprocessed image data as inputs across a trained machine learning model to generate output indicative of bone mineral density of the first bone;
determine a T-score value based on the bone mineral density of the first bone; and
provide, on a user interface, an output based on the T-score value.

18. The system according to claim 17, the program instructions further being executable to:
determine a Z-score value based on the bone mineral density of the first bone; and
provide, on the user interface, an output based on the Z-score value.

19. The system according to claim 17, the program instructions further being executable to:
determine a fracture risk based on the bone mineral density of the first bone; and
provide, on the user interface, an output based on the fracture risk assessment.

20. The system according to claim 17, the program instructions further being executable to:
determine a recommended surgical implant type based on the bone mineral density of the first bone or the T-score value; and
provide, on the user interface, an output based on the recommended surgical implant type.
